(19) United States
(12) Patent Application Publication
(10) Pub. No.: US 2014/0234415 A1
(43) Pub. Date: Aug. 21, 2014

(54) Tablet Dosage Forms

(71) Applicant: ABBVIE INC., WAUKEGAN, IL (US)

(72) Inventors: Todd S. McDermott, Libertyville, IL (US); Peter T. Mayer, Libertyville, IL (US); Benjamin M. Collman, Wilmette, IL (US); Weili Wang, Buffalo Grove, IL (US); Bei Chen, Buffalo Grove, IL (US)

(21) Appl. No.: 14/184,239

(22) Filed: Feb. 19, 2014

Related U.S. Application Data
(60) Provisional application No. 61/767,101, filed on Feb. 20, 2013.

(51) Int. Cl. A61K 9/24; B29C 43/20; A61K 31/7072; A61K 31/427; A61K 31/513

(52) U.S. Cl. CPC A61K 9/209; A61K 31/427; A61K 31/513; A61K 31/7072; B29C 43/203
USPC 424/465; 514/274; 514/50; 264/255

(57) ABSTRACT

The present invention features tablet dosage forms comprising two or more different active ingredients. In one embodiment, a tablet dosage form of the invention comprises a first layer and a second layer, wherein the first layer comprises polymer-based solid dispersion particles having a mean particle size of no more than 200 μm.

### CROSS-REFERENCE TO RELATED APPLICATION

This application claims priority to U.S. Provisional Application Ser. No. 61/767,101, filed Feb. 20, 2013, which is incorporated by reference in its entirety.

### FIELD OF THE INVENTION

The present invention relates to tablet dosage forms comprising two or more different active ingredients.

### BACKGROUND

Kaletra® (marketed as Aluvia® in certain developing countries) is one of the most affordable and successful HIV protease inhibitors in the world. Kaletra contains co-formulated lopinavir and ritonavir, in which lopinavir is the primary HIV protease inhibitor, and ritonavir functions as a pharmacokinetic booster. Upon administration, ritonavir suppresses the CYP3A4-mediated lopinavir metabolism, thereby increasing the plasma concentration of lopinavir.

Prior to 2005, Kaletra was only available in liquid forms in which lopinavir and ritonavir were dissolved in organic solvents. These liquid formulations are unstable at high temperatures and therefore often require refrigerated storage conditions. Moreover, these formulations need to be taken with food in order to provide adequate bioavailability. For patients residing in economically challenged or developing countries, these requirements represent a particularly challenging dilemma.

After intensive research and major formulation breakthroughs, a heat-stable Kaletra tablet was developed and approved in 2005. This heat-stable Kaletra tablet contains 200 mg of lopinavir and 50 mg of ritonavir, and provides critically important advantages for patients in developing countries, including storage without refrigeration, no dietary restrictions, and lower pill burden.

Lamivudine (3TC) is a potent nucleoside analog reverse transcriptase inhibitor, and is commercially available as Epivir® tablet. One form of Epivir tablet contains 150 mg of crystalline lamivudine. Lamivudine is often given in combination with zidovudine, with which it is highly synergistic. Zidovudine (AZT) is another nucleoside analog reverse-transcriptase inhibitor. Combivir® tablet is a fixed dose combination containing 150 mg of crystalline lamivudine and 300 mg of crystalline zidovudine.

### SUMMARY OF THE INVENTION

It has been particularly challenging to compress another layer of an active ingredient(s) to the Kaletra tablet core to make a bilayer tablet. Lopinavir and ritonavir are among the most difficult to be formulated in solid dosage forms. In order to provide sufficient bioavailability to lopinavir/ritonavir, a significant amount of polymers is used to form an amorphous solid dispersion in which lopinavir and ritonavir are molecularly dispersed. When prepared using melt extrusion, the extrudate, which comprises the amorphous solid dispersion of lopinavir and ritonavir, is often milled, and the milled solid dispersion particles are then compressed, together with other additives, to form a tablet core. The use of the solid dispersion technology leads to a large tablet size. For instance, a Kaletra tablet containing 200 mg lopinavir and 50 mg ritonavir has a weight of at least 1,200 mg.

A bilayer tablet often requires the two layers of active ingredients to be of similar sizes in order to provide manufacturability and/or physical stability. For a bilayer tablet that contains a Kaletra tablet core, this would require that the other layer of active ingredient(s) have a weight comparable to 1,200 mg. This would create a bilayer tablet that is too large to be orally administered.

On the other hand, reducing the size of the other layer of active ingredient(s) has led to less manufacturability and/or physical stability. For instance, multiple attempts to compress 200-300 mg of another layer of active ingredient(s) against a 1,200 mg lopinavir/ritonavir tablet core have failed to produce a physically stable bilayer tablet. The bilayer tablets so prepared often showed severe cracking. See, for example, FIG. 1.

It was surprisingly found that when the mean particle size of the solid dispersion particles used to prepare the lopinavir/ritonavir tablet core is reduced to less than 215 μm, the cracking in the bilayer tablets can be eliminated or significantly reduced. See, for example, FIG. 2.

Accordingly, in one aspect, the present invention features a tablet dosage form comprising a first layer and a second layer. The first layer comprises compressed solid dispersion particles each of which comprises ritonavir and lopinavir formulated in a solid dispersion, said solid dispersion comprising a pharmaceutically acceptable hydrophilic polymer and a pharmaceutically acceptable surfactant. The mean particle size of the solid dispersion particles is no more than 200 μm. Preferably, the mean particle size of the solid dispersion particles is no more than 190 μm. Also preferably, the mean particle size of the solid dispersion particles is no more than 180 μm. Also preferably, the mean particle size of the solid dispersion particles is from 120 to 200 μm. Also preferably, the mean particle size of the solid dispersion particles is from 120 to 190 μm. Also preferably, the mean particle size of the solid dispersion particles is from 120 to 180 μm. Also preferably, the mean particle size of the solid dispersion particles is from 140 to 160 μm. Also preferably, the mean particle size of the solid dispersion particles is from 160 to 180 μm. The second layer comprises another therapeutic agent, such as lamivudine or a combination of lamivudine and zidovudine.

The tablet dosage form preferably is a coated bilayer tablet.

The weight ratio of the second layer to the first layer preferably is no more than 1:2. More preferably, the weight ratio of the second layer to the first layer is no more than 1:3. Highly preferably, the weight ratio of the second layer to the first layer is no more than 1:4. Also highly preferably, the weight ratio of the second layer to the first layer is no more than 1:5. For example, the weight ratio of the second layer to the first layer is 1:5. For another example, the weight ratio of the second layer to the first layer is 1:6. For yet another example, the weight ratio of the second layer to the first layer is from 1:5 to 1:6.

The tablet dosage form preferably is no more than 1.6 g, and the first layer preferably is at least 1 g. For instance, the dosage form is no more than 1.6 g, and the first layer can be at least 1.1 g. For another instance, the dosage form is no more than 1.6 g, and the first layer can be at least 1.2 g. Preferably, the dosage form is from 1.4 to 1.6 g, and the first layer is from 1.1 to 1.3 g.

The first layer can constitute, for example, at least 60% by weight of the tablet dosage form. Preferably, the first layer constitutes at least 70% by weight of the tablet dosage form. More preferably, the first layer constitutes at least 75% by weight of the tablet dosage form. Also preferably, the first layer constitutes at least 85% by weight of the tablet dosage form.

Lopinavir and ritonavir in the first layer preferably are molecularly dispersed in the solid dispersion. Also preferably, the solid dispersion is an amorphous solid dispersion. More preferably, the solid dispersion is solid solution or glassy solution.

Lopinavir and ritonavir can, for example, be formulated in the same solid dispersion, solid solution or glassy solution. Lopinavir and ritonavir can also, for example, be separately formulated in different solid dispersions, solid solutions or glassy solutions, and then milled to the desired particle sizes and combined and compressed.

Suitable pharmaceutically acceptable polymers for use in the solid dispersion of the first layer preferably have a Tg of at least 50° C. The first layer preferably contains at least 50% by weight of such a pharmaceutically acceptable polymer or a combination of such polymers. Preferably, the first layer contains from 50 to 80% by weight of such a polymer or a combination of such polymers. Also preferably, the first layer contains from 60 to 80% by weight of such a polymer or a combination of such polymers. Highly preferably, the first layer contains from 70 to 75% by weight of such a polymer or a combination of such polymers. As used herein, the term “% by weight”, when used in describing the amount of a component in a tablet layer, refers to the weight percentage of that component in that layer. Preferred polymers include, but are not limited to, copovidone.

Suitable pharmaceutically acceptable surfactants for use in the solid dispersion of the first layer include non-ionic surfactants which preferably have an HLB value of from 4 to 10. The first layer preferably contains from 2 to 20% by weight of such a surfactant, or a combination of such surfactants, which has an HLB value of from 4 to 10. More preferably, the first layer contains from 5 to 15% by weight of such a surfactant, or a combination of such surfactants, which has an HLB value of from 4 to 10. Highly preferably, the first layer contains from 5 to 10% by weight of such a surfactant, or a combination of such surfactants, which has an HLB value of from 4 to 10. For example, the first layer can contain less than 10% by weight of such a surfactant, or a combination of such surfactants, which has an HLB value of from 4 to 10. Preferred surfactants include, but are not limited to, sorbitan monolaurate.

The second layer in a tablet dosage form of the invention comprises another therapeutic agent which is preferably mixed with other excipients. The therapeutic agent in the second layer can be formulated in solid dispersion, or in non-solid dispersion forms such as granules or physical mixtures with suitable excipients. The therapeutic agent in the second layer can be, for example and without limitation, in an amorphous form. The therapeutic agent can also be, for example and without limitation, in a crystalline form.

The therapeutic agent in the second layer can be, for example, an anti-HIV agent, such as a nucleoside reverse transcriptase inhibitor, a non-nucleoside reverse transcriptase inhibitor, a protease inhibitor, an integrase inhibitor, an entry or fusion inhibitor, or a combination thereof. Non-limiting examples of suitable anti-HCV agents include atazanavir, darunavir, amprenavir, fosamprenavir, indinavir, nelfinavir, saquinavir, tipranavir, lamivudine, abacavir, stavudine, didanosine, zidovudine, emtricitabine, tenofovir, delavirdine, efavirenz, rilpivirine, etravirine, nevirapine, enfuvirtide, maraviroc, raltegravir, and pharmaceutically acceptable salts thereof, and any combinations thereof. The therapeutic agent in the second layer preferably comprises lamivudine. Also preferably, the therapeutic agent in the second layer comprises lamivudine and zidovudine. Other therapeutic agents, such as anti-HCV agents, can also be used and formulated in the second layer. In one example, the second layer comprises (1) from 0% to 75% by weight of microcrystalline cellulose, (2) from 0% to 50% by weight of lactose, (3) from 0.5% to 5% by weight of sodium starch glycolate, (4) from 0.1% to 2% by weight of sodium stearyl fumarate; (5) from 0.2% to 3% by weight of colloidal silicon dioxide, and (6) from 0% to 75% by weight of hydroxypropyl cellulose.

In one embodiment, the first layer is 1.2 g, and the second layer is from 0.2 to 0.3 g.

In another embodiment, the first layer contains 200 mg lopinavir and 50 mg ritonavir, and the second layer preferably comprises 75 mg lamivudine.

In still another embodiment, the first layer contains 200 mg lopinavir and 50 mg ritonavir, and the second layer comprises 75 mg lamivudine and 150 mg zidovudine.

In yet another embodiment, the first layer comprises 200 mg lopinavir and 50 mg ritonavir, and the second layer comprises 200 mg raltegravir (or the corresponding amount of raltegravir salt, e.g., 217.2 mg raltegravir potassium).

In yet another embodiment, the first layer of a tablet dosage form of the invention comprises 200 mg lopinavir and 50 mg ritonavir, and the second layer comprises 75 mg lamivudine. With respect to lopinavir and lamivudine, the tablet dosage form is bioequivalent to a combination of a Kaletra tablet (200 mg lopinavir and 50 mg ritonavir per tablet) and one half of an Epivir tablet (150 mg lamivudine per tablet). Thus, with respect to lopinavir and lamivudine, two tablet dosage forms of this embodiment are bioequivalent to a combination of two Kaletra tablet (containing 200 mg lopinavir and 50 mg ritonavir per tablet) and one Epivir tablet (150 mg lamivudine per tablet). As used herein, a first dosage form is bioequivalent to a second dosage form (or a combination of dosage forms) with respect to an active ingredient if the 90% confidence intervals around the AUC_{∞} point estimate of the active ingredient in the first dosage form, relative to the second dosage form (or the combination of dosage forms), is within the range of from 80% to 125% when tested in a single dose study. A first dosage form is also considered bioequivalent to a second dosage form (or a combination of dosage forms) with respect to an active ingredient if the 90% confidence intervals around the AUCₜ point estimate of the active ingredient in the first dosage form, relative to the second dosage form (or the combination of dosage forms), is within the range of from 80% to 125% when tested in a single or multiple dose study. In a single dose study, t can be from 24 to 36 hours, such as 24 or 36 hours. In a multiple dose study, t is one complete dosing interval. AUC_{∞} or AUCₜ point estimates can be tested in either humans or dogs (e.g., beagles); preferably AUC_{∞} or AUCₜ point estimates are determined in humans.

In yet another embodiment, the first layer of a tablet dosage form of the invention comprises 200 mg lopinavir and 50 mg ritonavir, and the second layer comprises 75 mg lamivudine and 150 mg zidovudine. With respect to lopinavir, lamivudine and zidovudine, the tablet dosage form is bioequivalent to a combination of a Kaletra tablet (200 mg lopinavir and 50 mg ritonavir per tablet) and one half of a Combivir tablet (150 mg lamivudine and 300 mg zidovudine per tablet). Thus, two tablet dosage forms of this embodiment are bioequivalent to a combination of two Kaletra tablets (200 mg lopinavir and 50 mg ritonavir per tablet) and one Combivir tablet (150 mg lamivudine and 300 mg zidovudine per tablet).

In yet another embodiment, the first layer comprises 200 mg lopinavir and 50 mg ritonavir, and the second layer comprises 200 mg raltegravir (or the corresponding amount of raltegravir salt, e.g., 217.2 mg raltegravir potassium). With respect to lopinavir and raltegravir, the tablet dosage form is bioequivalent to a combination of a Kaletra tablet (200 mg lopinavir and 50 mg ritonavir per tablet) and one half of an Isentress® tablet (400 mg) (containing 434.4 mg raltegravir potassium per tablet). Thus, two tablet dosage forms of this embodiment are bioequivalent to a combination of two Kaletra tablets (200 mg lopinavir and 50 mg ritonavir per tablet) and one Isentress tablet (400 mg) (containing 434.4 mg raltegravir potassium per tablet).

Throughout this disclosure, features of a tablet dosage form of the invention may be described separately or in different examples, embodiments or preferences, e.g., the preferred mean particles sizes, the preferred weight ratios of the second layer over the first layer, the preferred pharmaceutically acceptable polymers or surfactants or their preferred amounts. The present invention contemplates tablet dosage forms with any combination of these separately described features.

In another aspect, the present invention features tablet dosage forms suitable for administration to pediatric patients. These pediatric tablet dosage forms often have the same composition as a tablet dosage form described herein, except that the amount of each ingredient is proportionally reduced. Preferably, the amount of each ingredient is reduced by half. For instance, the first layer of a pediatric tablet dosage form can comprise 100 mg lopinavir and 25 mg ritonavir, and the second layer comprises 37.5 mg lamivudine. For another instance, the first layer of a pediatric tablet dosage form can comprise 100 mg lopinavir and 25 mg ritonavir, and the second layer comprises 37.5 mg lamivudine and 75 mg zidovudine. Accordingly, each tablet dosage form of the invention can have a corresponding pediatric tablet dosage form in which the amount of each ingredient is reduced by half.

In yet another aspect, the present invention feature processes of making a tablet dosage form of the invention. The processes comprise compressing the first layer and the second layer into a bilayer.

In one embodiment, a process of the invention comprise (1) solidifying a homogeneous melt which comprises lopinavir, ritonavir, a pharmaceutically acceptable hydrophilic polymer as described herein and a pharmaceutically acceptable surfactant as described herein; (2) milling the solidified melt to a desired mean particle size as described herein; (3) adding the milled melt, optionally with other excipients, to a bilayer tablet press; (4) adding another pharmaceutical preparation comprising another therapeutic agent to the bilayer tablet press; and (5) compressing the milled melt and the other pharmaceutical preparation to form a bilayer tablet. The milled melt, optionally with the other excipients, forms the first layer of a tablet dosage form of the invention as described herein, while the other pharmaceutical preparation forms the second layer as described herein. Any tablet dosage form of the invention can be prepared according to this process. If needed, another layer(s) of active ingredient(s) can be further added.

In still another aspect, the present invention features a bottle of tablets of the invention, wherein no more than 10% of the tablets in the bottle show cracking. Preferably, no more than 5% of the tablets in the bottle show cracking. More preferably, no more than 2% of the tablets in the bottle show cracking. Highly preferably, no more than 1% of the tablets in the bottle show cracking. Most preferably, no tablet in the bottle shows cracking.

The present invention further features methods of treating HIV infection. The methods comprise administering a tablet dosage form of the invention to a HIV patient in need thereof.

In any aspect, preference, example or embodiment described herein, lopinavir and ritonavir in the first layer of a tablet dosage form of the invention can be readily replaced with another therapeutic agent(s). Therefore, the present invention also features such tablet dosage forms as well as their corresponding pediatric tablet dosage forms.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1A illustrates the severe crack in the coated bilayer tablets. Each of the tablets comprises a lopinavir/ritonavir layer and a lamivudine layer and is coated with Color Yellow. The lopinavir/ritonavir layer comprises 200 mg lopinavir and 50 mg ritonavir which are dispersed in amorphous solid dispersion composed of copovidone and sorbitan monolaurate. The mean particle size of the lopinavir/ritonavir solid dispersion is about 210 μm. The total weight of the lopinavir/ritonavir layer is about 1,200 mg. The lamivudine layer comprises 75 mg lamivudine and has a total weight of about 200 mg. At least 50% of such coated tablets showed severe cracks. X-ray tomography showed that these cracks were not the bilayer interface. Instead, they started under the bilayer interface and extended into the lopinavir/ritonavir layer.

FIG. 1B is an enlarged view of a tablet prepared as described in FIG. 1A. Cracking was shown, which extended into the lopinavir/ritonavir layer.

FIG. 2A shows coated bilayer tablets with the same composition as described in FIG. 1A, except that the mean particle size of the lopinavir/ritonavir solid dispersion was no more than 180 μm. No cracking showed in these tablets.

FIG. 2B is an enlarged view of a table prepared according to FIG. 2A. No cracking was observed.

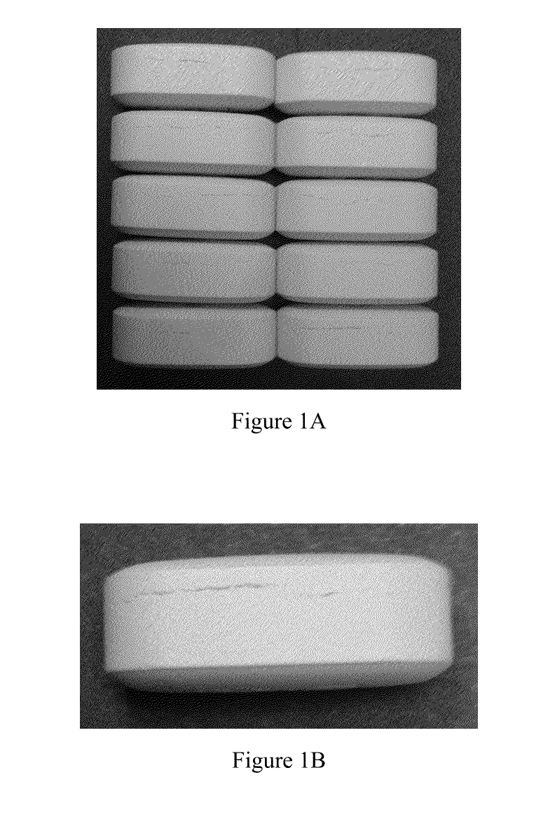

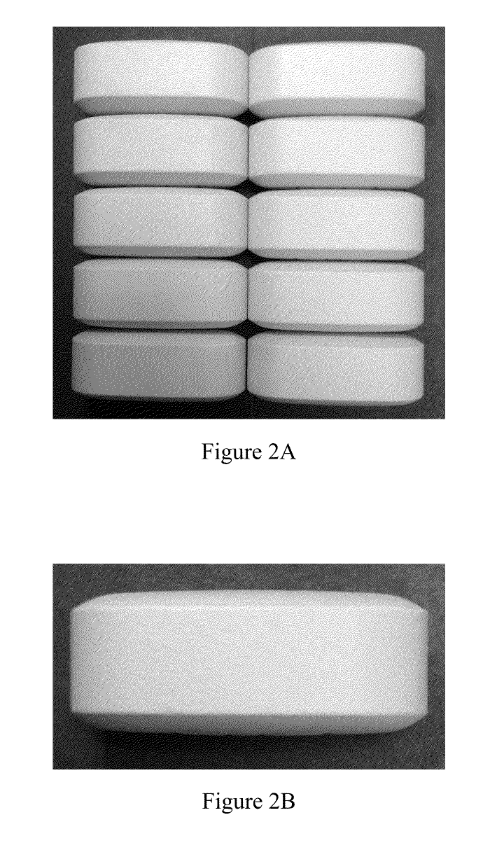

### DETAILED DESCRIPTION

Combining lopinavir/ritonavir with another therapeutic agent (e.g., lamivudine, or lamivudine/zidovudine) in a solid, fixed-dose combination formulation that is bioequivalent or substantially similar in pharmacokinetic profiles to their respective individual tablet forms have not been successful. For instance, Epivir and Combivir tablets are immediate release formulations and can be prepared by blending crystalline lamivudine and zidovudine with other excipients. In contrast, Kaletra tablet is an erosion-based formulation and is prepared by dispersing lopinavir and ritonavir in a polymer-based matrix to form an amorphous solid dispersion. Therefore, when these two formulations are mixed together, the release profile of each active ingredient is expected to be significantly altered, thereby producing a composition that is unlikely bioequivalent or substantially similar to a combination of the respective individual tablet forms.

Combining Epivir or Combivir with Kaletra into a single, bilayer tablet has also been found exceedingly difficult. Kaletra tablet has a large tablet size due to the use of a large amount of polymers in order to confer adequate bioavailability to lopinavir and ritonavir. For instance, a Kaletra tablet containing 200 mg lopinavir and 50 mg ritonavir has a weight of at least 1,200 mg. A bilayer tablet often requires the two layers of active ingredients to be of similar sizes in order to provide manufacturability and/or physical stability. This would require the lamivudine or lamivudine/zidovudine layer to have a weight comparable to 1,200 mg, which would make the final product too large to be orally administered.

On the other hand, reducing the size of the lamivudine or lamivudine/zidovudine layer would lead to less manufacturability and/or physical stability. For instance, many attempts to compress a 200-300 mg lamivudine layer (containing 75 mg lamivudine) against a 1,200 mg lopinavir/ritonavir tablet core (containing 200 mg lopinavir and 50 mg ritonavir) have failed to produce a stable bilayer tablet.

It was unexpectedly found that when the mean particle size of the solid dispersion particles used to prepare the lopinavir/ritonavir tablet core is reduced to 200 μm or less, the cracking in the bilayer tablets can be eliminated or significantly reduced. Cf Example 3 of U.S. Pat. No. 8,025,899, which describes a post-extrusion process comprising milling the extrudates (which include a solid dispersion of lopinavir and ritonavir) to a particle size of about 250 μm, followed by compressing the milled extrudates into a tablet.

Accordingly, in one aspect, the present invention features a tablet dosage form comprising a first layer and a second layer. The first layer comprises compressed solid dispersion particles, each of which comprises ritonavir and lopinavir formulated in a solid dispersion, and said solid dispersion comprising a pharmaceutically acceptable hydrophilic polymer and a pharmaceutically acceptable surfactant. The mean particle size of these solid dispersion particles is no more than 200 μm. Preferably, the mean particle size of these solid dispersion particles is no more than 190 μm. More preferably, the mean particle size of these solid dispersion particles is no more than 180 μm. Also preferably, the mean particle size of the solid dispersion particles is from 100 to 190 μm, such as from 110 to 190 μm, from 120 to 190 μm, from 130 to 190 μm, from 140 to 190 μm, from 150 to 190 μm, or from 160 to 190 μm. Also preferably, the mean particle size of the solid dispersion particles is from 100 to 180 μm, such as from 110 to 180 μm, from 120 to 180 μm, from 130 to 180 μm, from 140 to 180 μm, from 150 to 180 μm, or from 160 to 180 μm. Also preferably, the mean particle size of the solid dispersion particles is from 100 to 170 μm, such as from 110 to 170 μm, from 120 to 170 lam, from 130 to 170 μm, from 140 to 170 μm, or from 150 to 170 μm. Also preferably, the mean particle size of the solid dispersion particles is from 100 to 160 μm, such as from 110 to 160 μm, from 120 to 160 μm, from 130 to 160 μm, or from 140 to 160 μm.

The particle size distribution can be determined by sieve analysis or other means as appreciated by those skilled in the art. An exemplary sieve analysis involves the use of sieves with different opening sizes. The sieves can be assembled into a column with the top sieve having the widest openings and each lower sieve in the column having smaller openings than the one above. The column can be placed in a mechanical shaker. The shaker shakes the column for a pre-determined amount of time. After the shaking is complete, the material on each sieve is weighed. The weight of the sample of each sieve is then divided by the total weight to give a percentage retained on each sieve. The mean particle size (MPS) can be calculated using the following equation:

$$M P S = \frac{{C}_{1} - 50 \%}{{C}_{1} - {C}_{2}} \times ( {S}_{2} - {S}_{1} ) + {S}_{1}$$

where C₁ is the accumulated percentage of particles just larger than 50%; C₂ is the accumulated percentage of particles just smaller than 50%; S₁ is the size of particles which has an accumulated percentage just larger than 50%, and S₂ is the size of the particles which have an accumulated percentage just smaller than 50%.

The second layer in a tablet dosage form of this aspect of the invention comprises another therapeutic agent, such as lamivudine or a combination of lamivudine and zidovudine. The therapeutic agent preferably is mixed with suitable excipients. The therapeutic agent in the second layer can be formulated in solid dispersion, or in non-solid dispersion forms such as granules or physical mixtures with other excipients. The therapeutic agent in the second layer can be, for example and without limitation, in an amorphous form. The therapeutic agent can also be, for example and without limitation, in a crystalline form.

A tablet dosage form of this aspect of the invention preferably is a coated bilayer tablet. The tablet dosage form can also include a third layer of yet another active ingredient, or two or more layers of other active ingredients.

The weight ratio of the second layer to the first layer according to this aspect of the invention preferably is no more than 1:2. More preferably, the weight ratio of the second layer to the first layer is no more than 1:3. Highly preferably, the weight ratio of the second layer to the first layer is no more than 1:4. Also highly preferably, the weight ratio of the second layer to the first layer is no more than 1:5. For example, the weight ratio of the second layer to the first layer is 1:5. For another example, the weight ratio of the second layer to the first layer is 1:6. For yet another example, the weight ratio of the second layer to the first layer is from 1:3 to 1:6. For yet another example, the weight ratio of the second layer to the first layer is from 1:4 to 1:6. For yet another example, the weight ratio of the second layer to the first layer is from 1:5 to 1:6.

A tablet dosage form of this aspect of the invention can be, for example and without limitation, from 1.3 g to 1.7 g, and the first layer can be, for example and without limitation, from 0.9 g to 1.2 g. For instance, the tablet dosage form can be from 1.3 g to 1.5 g, and the first layer is from 1.0 g to 1.2 g. For instance, the tablet dosage form can be from 1.4 g to 1.6 g, and the first layer is from 1.1 g to 1.3 g. In one example, the tablet dosage form is no more than 1.7 g, and the first layer is at least 1 g. In another example, the tablet dosage form can be no more than 1.6 g, and the first layer is at least 1 g. In another yet example, the tablet dosage form can be no more than 1.5 g, and the first layer is at least 1 g. In yet another example, the tablet dosage form is no more than 1.7 g, and the first layer is at least 1.1 g. In yet another example, the tablet dosage form is no more than 1.6 g, and the first layer is at least 1.1 g. In yet another example, the tablet dosage form is no more than 1.5 g, and the first layer is at least 1.1 g. In yet another example, the tablet dosage form is no more than 1.7 g, and the first layer is at least 1.2 g. In yet another example, the tablet dosage form is no more than 1.6 g, and the first layer is at least 1.2 g. In yet another example, the tablet dosage form is no more than 1.5 g, and the first layer is at least 1.2 g. In yet another example, the tablet dosage form is no more than 1.7 g, and the first layer is from 1.0 g to 1.3 g. In another example, the tablet dosage form is no more than 1.7 g, and the first layer is at from 1.0 g to 1.2 g. In yet another example, the tablet dosage form is no more than 1.7 g, and the first layer is from 1.0 g to 1.1 g. In yet another example, the tablet dosage form is no more than 1.7 g, and the first layer is from 1.1 g to 1.3 g. In yet another example, the tablet dosage form is no more than 1.7 g, and the first layer is from 1.1 g to 1.2 g. In yet another example, the tablet dosage form is no more than 1.6 g, and the first layer is from 1.0 g to 1.3 g. In another example, the tablet dosage form is no more than 1.6 g, and the first layer is at from 1.0 g to 1.2 g. In yet another example, the tablet dosage form is no more than 1.6 g, and the first layer is from 1.0 g to 1.1 g. In yet another example, the tablet dosage form is no more than 1.6 g, and the first layer is from 1.1 g to 1.3 g. In yet another example, the tablet dosage form is no more than 1.6 g, and the first layer is from 1.1 g to 1.2 g. In yet another example, the tablet dosage form is no more than 1.5 g, and the first layer is from 1.0 g to 1.3 g. In another example, the tablet dosage form is no more than 1.5 g, and the first layer is at from 1.0 g to 1.2 g. In yet another example, the tablet dosage form is no more than 1.5 g, and the first layer is from 1.0 g to 1.1 g. In yet another example, the tablet dosage form is no more than 1.5 g, and the first layer is from 1.1 g to 1.3 g. In yet another example, the tablet dosage form is no more than 1.5 g, and the first layer is from 1.1 g to 1.2 g.

Preferably, the dosage form is from 1.4 to 1.6 g, and the first layer is from 1.1 to 1.3 g. Also preferably, the dosage form is from 1.4 to 1.6 g, and the first layer is from 1.2 to 1.3 g.

The first layer can comprise, for example, at least 60% by weight of the tablet dosage form. Preferably, the first layer comprises at least 70% by weight of the tablet dosage form. More preferably, the first layer comprises at least 75% by weight of the tablet dosage form. Also preferably, the first layer comprises at least 85% by weight of the tablet dosage form. In one example, the first layer comprises from 60 to 90% by weight of the tablet dosage form. In another example, the first layer comprises from 70 to 90% by weight of the tablet dosage form. In another example, the first layer comprises from 80 to 90% by weight of the tablet dosage form. In another example, the first layer comprises from 60 to 80% by weight of the tablet dosage form. In another example, the first layer comprises from 70 to 80% by weight of the tablet dosage form.

Lopinavir and ritonavir in the first layer preferably are molecularly dispersed in the solid dispersion. Also preferably, the solid dispersion is an amorphous solid dispersion. More preferably, the solid dispersion is solid solution or glassy solution.

Lopinavir and ritonavir can, for example, be formulated in the same solid dispersion, solid solution or glassy solution. Lopinavir and ritonavir can also, for example, be separately formulated in different solid dispersions, solid solutions or glassy solutions, and then milled to the desired particle sizes and mixed.

In one embodiment, the first layer preferably includes a solid dispersion, where the solid dispersion comprises ritonavir, lopinavir, the pharmaceutically acceptable hydrophilic polymer, and the pharmaceutically acceptable surfactant.

In another embodiment, the first layer comprises a solid solution, where the solid solution comprises ritonavir, lopinavir, the hydrophilic polymer, and the surfactant.

In still another embodiment, the first layer comprises a glassy solution, where the glassy solution comprises ritonavir, lopinavir, the hydrophilic polymer, and the surfactant.

In yet another embodiment, the first layer comprises a first and second solid dispersions (i.e., a lopinavir solid dispersion and a ritonavir solid dispersion, respectively), wherein the first solid dispersion comprises lopinavir and a first pharmaceutically acceptable hydrophilic polymer, and the second solid dispersion comprises ritonavir and a second pharmaceutically acceptable hydrophilic polymer, and the first layer also contains a pharmaceutically acceptable surfactant. The first and second hydrophilic polymers can be the same or different.

In yet another embodiment, the first layer comprises a first and second solid dispersions (i.e., a lopinavir solid dispersion and a ritonavir solid dispersion, respectively), wherein the first solid dispersion comprises lopinavir, a first pharmaceutically acceptable hydrophilic polymer, and a first pharmaceutically acceptable surfactant, and the second solid dispersion comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In yet another embodiment, the first layer comprises a first and second solid solutions (i.e., a lopinavir solid solution and a ritonavir solid solution, respectively), wherein the first solid solution comprises lopinavir and a first pharmaceutically acceptable hydrophilic polymer, and the second solid solution comprises ritonavir and a second pharmaceutically acceptable hydrophilic polymer, and the first layer also contains a pharmaceutically acceptable surfactant. The first and second hydrophilic polymers can be the same or different.

In yet another embodiment, the first layer comprises a first and second solid solutions (i.e., a lopinavir solid solution and a ritonavir solid solution, respectively), wherein the first solid solution comprises lopinavir, a first pharmaceutically acceptable hydrophilic polymer, and a first pharmaceutically acceptable surfactant, and the second solid solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In still another embodiment, the first layer comprises a first and second glassy solutions (i.e., a lopinavir glassy solution and a ritonavir glassy solution, respectively), wherein the first glassy solution comprises lopinavir and a first pharmaceutically acceptable hydrophilic polymer, and the second glassy solution comprises ritonavir and a second pharmaceutically acceptable hydrophilic polymer, and the first layer also contains a pharmaceutically acceptable surfactant. The first and second hydrophilic polymers can be the same or different.

In yet another embodiment, the first layer comprises a first and second glassy solutions (i.e., a lopinavir glassy solution and a ritonavir glassy solution, respectively), wherein the first glassy solution comprises lopinavir, a first pharmaceutically acceptable hydrophilic polymer, and a first pharmaceutically acceptable surfactant, and the second glassy solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

A solid dispersion employed in this aspect of the invention preferably comprises or consists of a single-phase (defined in thermodynamics) in which lopinavir and/or ritonavir, together with the pharmaceutically acceptable hydrophilic polymer and/or the pharmaceutically acceptable surfactant, are molecularly dispersed. In such cases, thermal analysis of the solid dispersion using differential scanning calorimetry (DSC) typically shows only one single glass transition temperature (T_{g}), and the solid dispersion does not contain any detectable crystalline lopinavir or ritonavir as measured by X-ray powder diffraction spectroscopy.

In one embodiment of this aspect of the invention, the first layer contains 200 mg lopinavir and 50 mg ritonavir, and the second layer comprises 75 mg lamivudine.

In still another embodiment, the first layer contains 200 mg lopinavir and 50 mg ritonavir, and the second layer comprises 75 mg lamivudine and 150 mg zidovudine.

In yet another embodiment, the first layer comprises 200 mg lopinavir and 50 mg ritonavir, and the second layer comprises 200 mg raltegravir (or the corresponding amount of raltegravir salt, e.g., 217.2 mg raltegravir potassium).

In yet another embodiment, the first layer of a tablet dosage form of the invention comprises 200 mg lopinavir and 50 mg ritonavir, and the second layer comprises 75 mg lamivudine. With respect to lopinavir and lamivudine, the tablet dosage form is bioequivalent to a combination of a Kaletra tablet (200 mg lopinavir and 50 mg ritonavir per tablet) and one half of an Epivir tablet (150 mg lamivudine per tablet). Thus, with respect to lopinavir and lamivudine, two tablet dosage forms of this embodiment are bioequivalent to a combination of two Kaletra tablet (containing 200 mg lopinavir and 50 mg ritonavir per tablet) and one Epivir tablet (150 mg lamivudine per tablet).

In yet another embodiment, the first layer of a tablet dosage form of the invention comprises 200 mg lopinavir and 50 mg ritonavir, and the second layer comprises 75 mg lamivudine and 150 mg zidovudine. With respect to lopinavir, lamivudine and zidovudine, the tablet dosage form is bioequivalent to a combination of a Kaletra tablet (200 mg lopinavir and 50 mg ritonavir per tablet) and one half of a Combivir tablet (150 mg lamivudine and 300 mg zidovudine per tablet). Thus, two tablet dosage forms of this embodiment are bioequivalent to a combination of two Kaletra tablets (200 mg lopinavir and 50 mg ritonavir per tablet) and one Combivir tablet (150 mg lamivudine and 300 mg zidovudine per tablet).

In yet another embodiment, the first layer comprises 200 mg lopinavir and 50 mg ritonavir, and the second layer comprises 200 mg raltegravir (or the corresponding amount of raltegravir salt, e.g., 217.2 mg raltegravir potassium). With respect to lopinavir and raltegravir, the tablet dosage form is bioequivalent to a combination of a Kaletra tablet (200 mg lopinavir and 50 mg ritonavir per tablet) and one half of an Isentress® tablet (400 mg) (containing 434.4 mg raltegravir potassium per tablet). Thus, two tablet dosage forms of this embodiment are bioequivalent to a combination of two Kaletra tablets (200 mg lopinavir and 50 mg ritonavir per tablet) and one Isentress tablet (400 mg) (containing 434.4 mg raltegravir potassium per tablet).

In another embodiment, a tablet dosage form comprises a first layer and a second layer, wherein the first layer comprises 200 mg lopinavir and 50 mg ritonavir and constitutes at least 70% by weight of the total dosage form, and the second layer comprises 75 mg lamivudine. The first layer comprises (i) at least 50% by weight a pharmaceutically acceptable hydrophilic polymer, and (ii) a pharmaceutically acceptable surfactant. The total weight of the tablet is no more than 1700 mg. Preferably, the tablet is no more than 1600 mg. More preferably, the tablet is no more than 1500 mg.

In yet another embodiment, a tablet dosage form comprises a first layer and a second layer, wherein the first layer comprises 200 mg lopinavir and 50 mg ritonavir and constitutes at least 70% by weight of the total dosage form, and the second layer comprises 75 mg lamivudine and 150 mg zidovudine. The first layer comprises (i) at least 50% by weight a pharmaceutically acceptable hydrophilic polymer, and (ii) a pharmaceutically acceptable surfactant. The total weight of the tablet is no more than 1700 mg. Preferably, the tablet is no more than 1600 mg.

A pharmaceutically acceptable salt of lopinavir or a pharmaceutically acceptable salt of ritonavir can be used, instead of lopinavir or ritonavir, respectively, in the first layer.

The other therapeutic agent in the second layer can be, for example and without limitation, an anti-HIV agent, an anti-HCV agent, or another anti-viral agent. Non-limiting examples of the other therapeutic agent include anti-HIV agents, such as lamivudine, zidovudine, didanosine, abacavir, efavirenz, emtricitabine, tenofovir, stavudine, delavirdine, rilpivirine, etravirine, nevirapine, enfuvirtide, maraviroc, raltegravir, dolutegravir, lersivirine, atazanavir, darunavir, amprenavir, fosamprenavir, indinavir, nelfinavir, saquinavir, tipranavir, or a pharmaceutically acceptable salt thereof, or a combination of two or more of the above agents or their respective salts (e.g., a combination of lamivudine and zidovudine, a combination of abacavir, lamivudine and zidovudine, a combination of efavirenz, emtricitabine and tenofovir, a combination of emtricitabine and tenofovir, or a combination of emtricitabine, rilpivirine and tenofovi). Other non-limiting examples of the other therapeutic agent in the second layer include anti-HCV agents, such as NM-811 (Novartis), SCY-635 (Scynexis), ITX-4520 (iTherx), ITX-5061 (iTherx), ANA-773 (Anadys), ABT-072 (Abbott), ABT-333 (Abbott), ANA-598 (Anadys), setrobuvir, BI-207127 (Boehringer Ingelheim), BILB-1941 (Boehringer Ingelheim), BMS-791325 (BMS), filibuvir, GL59728 (Glaxo), GL60667 (Glaxo), GS-9669 (Gilead), IDX-375 (Idenix), MK-3281 (Merck), tegobuvir, TMC-647055 (Tibotec), VCH-759 (Vertex & ViraChem), VCH-916 (ViraChem), VX-222 (VCH-222) (Vertex & ViraChem), VX-759 (Vertex), ACH-2928 (Achillion), AZD2836 (Astra-Zeneca), AZD7295 (Astra-Zeneca), BMS-790052 (BMS), BMS-824393 (BMS), EDP-239 (Enanta), GS-5885 (Gilead), PPI-1301 (Presidio), PPI-461 (Presidio), GS-6620 (Gilead), IDX-102 (Idenix), IDX-184 (Idenix), INX-189 (Inhibitex), MK-0608 (Merck), PSI-7977 (Pharmasset), PSI-938 (Pharmasset), RG7128 (Roche), TMC64912 (Medivir), GSK625433 (GlaxoSmithKline), BCX-4678 (BioCryst), ABT-450 (Abbott/Enanta), ACH-1095 (Achillion), ACH-1625 (Achillion), ACH-2684 (Achillion), AVL-181 (Avila), AVL-192 (Avila), BI-201335 (Boehringer Ingelheim), BMS-650032 (BMS), boceprevir, danoprevir, GS-9132 (Gilead), GS-9256 (Gilead), GS-9451 (Gilead), IDX-136 (Idenix), IDX-316 (Idenix), IDX-320 (Idenix), MK-5172 (Merck), narlaprevir, PHX-1766 (Phenomix), telaprevir, TMC-435 (Tibotec), vaniprevir, VBY708 (Virobay), VX-500 (Vertex), VX-813 (Vertex), VX-985 (Vertex), CTS-1027 (Conatus), GS-9620 (Gilead), PF-4878691 (Pfizer), R05303253 (Roche), ALS-2200, ALS-2158, GSK62336805, or a pharmaceutically acceptable salt thereof, or a combination of two or more of the above agents or their respective salts.

The amount of the other therapeutic agent in the second layer can range, for example and without limitation, from 10 mg to 300 mg, such as from 50 mg to 200 mg.

Preferably, the other therapeutic agent in the second layer is a HIV reverse transcriptase inhibitors (e.g., lamivudine or zidovudine), a HIV integrase (e.g., raltegravir or dolutegravir), a HIV entry inhibitor (e.g., enfuvirtide or maraviroc), or a combination thereof (e.g., a combination of lamivudine and zidovudine, a combination of abacavir, lamivudine and zidovudine, a combination of efavirenz, emtricitabine and tenofovir, a combination of emtricitabine and tenofovir, or a combination of emtricitabine, rilpivirine and tenofovir). More preferably, the other therapeutic agent in the second layer comprises lamivudine. In one embodiment, the second layer comprises, in addition to the other therapeutic agent as described above, (1) from 0% to 75% by weight of microcrystalline cellulose, (2) from 0% to 50% by weight of lactose, (3) from 0.5% to 5% by weight of sodium starch glycolate, (4) from 0.1% to 2% by weight of sodium stearyl fumarate; (5) from 0.2% to 3% by weight of colloidal silicon dioxide, and (6) from 0% to 75% by weight of hydroxypropyl cellulose.

A pharmaceutically acceptable hydrophilic polymers employed in the first layer of a tablet dosage form of the invention preferably have a T_{g} of at least 50° C. More preferably, the hydrophilic polymer has a T_{g} of at least 60° C., such as at least 80° C., at least 100° C., or from 80° C. to 180° C., or from 100° C. to 150° C. Hydrophilic polymers with a T_{g} as described above allow for the preparation of solid dispersions that are mechanically stable and, within ordinary temperature ranges, sufficiently temperature stable. Hydrophilic polymers having a T_{g} of below 50° C. may also be included.

A pharmaceutically acceptable hydrophilic polymer employed in the first layer of a tablet dosage form of the invention preferably is water-soluble. The first layer may also include a poorly water-soluble or water-insoluble polymer, such as a cross-linked polymer. A hydrophilic polymer employed in the first layer preferably has an apparent viscosity, when dissolved at 20° C. in an aqueous solution at 2% (w/v), of 1 to 5000 mPa·s., and more preferably of 1 to 700 mPa·s, and most preferably of 5 to 100 mPa·s.

Pharmaceutically acceptable hydrophilic polymers that are suitable for use in the first layer include, but are not limited to, homopolymers or copolymers of N-vinyl lactams, such as homopolymers or copolymers of N-vinyl pyrrolidone (e.g., polyvinylpyrrolidone (PVP), or copolymers of N-vinyl pyrrolidone and vinyl acetate or vinyl propionate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); high molecular polyalkylene oxides, such as polyethylene oxide, polypropylene oxide, and copolymers of ethylene oxide and propylene oxide; polyacrylates or polymethacrylates, such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), and poly(hydroxyalkyl methacrylates); polyacrylamides; vinyl acetate polymers, such as copolymers of vinyl acetate and crotonic acid, and partially hydrolyzed polyvinyl acetate (also referred to as partially saponified “polyvinyl alcohol”); polyvinyl alcohol; oligo- or polysaccharides, such as carrageenans, galactomannans, and xanthan gum; polyhydroxyalkylacrylates; polyhydroxyalkyl-methacrylates; copolymers of methyl methacrylate and acrylic acid; polyethylene glycols (PEGs); or any mixture thereof.

Non-limiting examples of suitable hydrophilic polymers include polyvinylpyrrolidone (PVP) K17, PVP K25, PVP K30, PVP K90, hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, HPMC P 55, Ethocel 4, Ethocel 7, Ethocel 10, Ethocel 14, Ethocel 20, copovidone (vinylpyrrolidone-vinyl acetate copolymer 60/40), polyvinyl acetate, methacrylate/methacrylic acid copolymer (Eudragit®) L100-55, Eudragit L100, Eudragit S100, polyethylene glycol (PEG) 400, PEG 600, PEG 1450, PEG 3350, PEG 4000, PEG 6000, PEG 8000, poloxamer 124, poloxamer 188, poloxamer 237, poloxamer 338, and poloxamer 407.

Of these, homopolymers or copolymers of N-vinyl pyrrolidone, such as copolymers of N-vinyl pyrrolidone and vinyl acetate, are preferred. A non-limiting example of a preferred polymer is a copolymer of 60% by weight of N-vinyl pyrrolidone and 40% by weight of vinyl acetate. Other preferred polymers include, without limitation, hydroxypropyl methylcellulose (HPMC, also known as hypromellose in USP), such as hydroxypropyl methylcellulose grade E5 (HPMC-E5); and hydroxypropyl methylcellulose acetate succinate (HPMC-AS).

Preferably, the first layer in a tablet dosage form of the invention comprises at least 50% by weight of a pharmaceutical acceptable hydrophilic polymer or polymers. More preferably, the first layer comprises at least 60% by weight of a pharmaceutically acceptable hydrophilic polymer or polymers. Highly preferably, the first layer comprises at least 70% weight of a pharmaceutically acceptable hydrophilic polymer or polymers. In one embodiment, the first layer comprises from 50 to 80% weight of a pharmaceutically acceptable hydrophilic polymer or polymers. In another embodiment, the first layer comprises from 60 to 80% weight of a pharmaceutically acceptable hydrophilic polymer or polymers. In another embodiment, the first layer comprises from 65 to 75% weight of a pharmaceutically acceptable hydrophilic polymer or polymers. In another embodiment, the first layer comprises from 70 to 75% weight of a pharmaceutically acceptable hydrophilic polymer or polymers.

In each aspect, example, embodiment and preference described herein, sugar alcohols or other binders may be used in addition to, or in lieu of, hydrophilic polymers in the first layer.

A pharmaceutically acceptable surfactant employed in the first layer of a tablet dosage form of the invention preferably is a non-ionic surfactant. More preferably, a pharmaceutically acceptable, non-ionic surfactant employed in the first layer has an HLB value of from 4 to 10. For instance, the surfactant can have an HLB value of from 7 to 9. A surfactant having an HLB value other than 4-10, such as a surfactant having an HLB value of below or above 10, may also be used. As used herein, a surfactant encompasses a mixture or combination of two or more different surfactants.

Pharmaceutically acceptable surfactants that are suitable for use in the first layer include, but are not limited to, polyoxyethylene alkyl ether, polyoxyethylene alkylaryl ether, polyethylene glycol fatty acid ester, alkylene glycol fatty acid mono ester, sucrose fatty acid ester, and sorbitan fatty acid mono ester. Non-limiting examples of suitable surfactants include polyoxyethylene (3) lauryl ether, polyoxyethylene (5) cetyl ether, polyoxyethylene (2) stearyl ether, polyoxyethylene (5) stearyl ether, polyoxyethylene (2) nonylphenyl ether, polyoxyethylene (3) nonylphenyl ether, polyoxyethylene (4) nonylphenyl ether, polyoxyethylene (3) octylphenyl ether, PEG-200 monolaurate, PEG-200 dilaurate, PEG-300 dilaurate, PEG-400 dilaurate, PEG-300 distearate, PEG-300 dioleate, propylene glycol monolaurate (e.g., Lauroglycol®); sucrose monostearate, sucrose distearate, sucrose monolaurate, sucrose dilaurate, sorbitan mono laurate (e.g., Span® 20), sorbitan monooleate, sorbitan monopalnitate (e.g., Span® 40), sorbitan stearate, or mixtures of one or more thereof.

The sorbitan mono fatty acid esters are preferred, with sorbitan mono laurate and sorbitan mono palmitate being particularly preferred.

Other pharmaceutically acceptable surfactants that may be used in the first layer include, but are not limited to, polyoxyethylene castor oil derivatives, e.g. polyoxyethyleneglycerol triricinoleate or polyoxyl 35 castor oil (Cremophor® EL; BASF Corp.) or polyoxyethyleneglycerol oxystearate such as polyethyleneglycol 40 hydrogenated castor oil (Cremophor® RH 40) or polyethyleneglycol 60 hydrogenated castor oil (Cremophor® RH 60); or block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, Poloxamer® 407 (BASF Wyandotte Corp.); or a mono fatty acid ester of polyoxyethylene (20) sorbitan, e.g. polyoxyethylene (20) sorbitan mono oleate (Tween® 80), polyoxyethylene (20) sorbitan monostearate (Tween® 60), polyoxyethylene (20) sorbitan mono palmitate (Tween® 40), polyoxyethylene (20) sorbitan monolaurate (Tween® 20).

In one embodiment, the first layer comprises two or more surfactants, wherein the surfactant(s) having an HLB value of from 4 to 10 accounts for at least 50% by weight, preferably at least 60% by weight, of the total amount of surfactants used in the first layer.

The first layer preferably comprises at least 1% by weight of a pharmaceutically acceptable surfactant. More preferably, the first layer comprises at least 2% by weight of a pharmaceutically acceptable surfactant. Highly preferably, the first layer comprises at least 5% by weight of a pharmaceutically acceptable surfactant. In one embodiment, the first layer comprise from 2% to 20% by weight of a pharmaceutically acceptable surfactant. In another embodiment, the first layer comprise from 4% to 20% by weight of a pharmaceutically acceptable surfactant. In another embodiment, the first layer comprise from 5% to 15% by weight of a pharmaceutically acceptable surfactant. In another embodiment, the first layer comprise from 5% to 10% by weight of a pharmaceutically acceptable surfactant. In yet another embodiment, the first layer contains from 2 to 20% by weight of a pharmaceutically acceptable surfactant, or a combination of such surfactants, which has an HLB value of from 4 to 10. In another embodiment, the first layer contains from 5 to 15% by weight of a pharmaceutically acceptable surfactant, or a combination of such surfactants, which has an HLB value of from 4 to 10. In another embodiment, the first layer contains from 5 to 10% by weight of a pharmaceutically acceptable surfactant, or a combination of such surfactants, which has an HLB value of from 4 to 10.

The first layer can, for example and without limitation, contain less than 10% by weight of a pharmaceutically acceptable surfactant, or a combination of such surfactants, which has an HLB value of from 4 to 10.

Features of a tablet dosage form of the invention may be described separately or in different aspects, examples, embodiments or preferences, e.g., the preferred mean particles sizes, the preferred weight ratios of the second layer over the first layer, the preferred pharmaceutically acceptable polymers or surfactants or their preferred amounts. However, the present invention contemplates tablet dosage forms with any combination of these features of different aspects, embodiments, examples or preferences.

In one embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.7 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer).

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.6 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a T_{g} of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer).

In another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.5 g, and the first layer is at least 1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer).

In still another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.5 g, and the first layer is at least 1.1 g. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer).

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.6 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.6 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.6 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.5 g, and the weight ratio of the first layer over the second layer is no less than 3:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.5 g, and the weight ratio of the first layer over the second layer is no less than 4:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

In yet another embodiment, a tablet dosage form of the invention comprises a first layer and a second layer, wherein the first layer comprises a pharmaceutically acceptable hydrophilic polymer, a pharmaceutically acceptable surfactant, 50 mg ritonavir and 200 mg lopinavir, in solid dispersion particles with a desired mean particle size as described herein, and wherein the second layer comprises another therapeutic agent (e.g., 75 mg lamivudine, or a combination of 75 mg lamivudine and 150 mg zidovudine). The tablet is no more than 1.5 g, and the weight ratio of the first layer over the second layer is no less than 5:1. Preferably, lopinavir and ritonavir in the first layer are formulated in solid dispersion comprising the hydrophilic polymer and the surfactant; more preferably, lopinavir and ritonavir in the first layer are formulated in solid solution comprising the hydrophilic polymer and the surfactant; highly preferably, lopinavir and ritonavir in the first layer are formulated in glassy solution comprising the hydrophilic polymer and the surfactant. The hydrophilic polymer preferably has a Tg of at least 50° C.; and more preferably, the hydrophilic polymer is copovidone. The surfactant preferably has a HLB value of from 4 to 10; and more preferably, the surfactant is sorbitan mono laurate. The first layer preferably comprises at least 50% by weight of the hydrophilic polymer (e.g., from 65 to 75% by weight of the hydrophilic polymer). The first layer is also preferably at least 1.1 g.

Where the first layer comprises 200 mg lopinavir and the second layer comprises 75 mg lamivudine, the tablet dosage form preferably is bioequivalent, with respect to lopinavir and lamivudine, to a combination of a Kaletra tablet (200 mg lopinavir and 50 mg ritonavir per tablet) and one half of an Epivir tablet (150 mg lamivudine per tablet); or two such tablet dosage forms preferably are bioequivalent, with respect to lopinavir and lamivudine, to a combination of two Kaletra tablets (200 mg lopinavir and 50 mg ritonavir per tablet) and one Epivir tablet (150 mg lamivudine per tablet). Where the first layer comprises 200 mg lopinavir and the second layer comprise a combination of 75 mg lamivudine and 150 mg zidovudine, the tablet dosage form preferably is bioequivalent, with respect to lopinavir, lamivudine and zidovudine, to a combination of a Kaletra tablet (200 mg lopinavir and 50 mg ritonavir per tablet) and one half of an Combivir tablet (150 mg lamivudine and 300 mg zidovudine per tablet); or two such tablet dosage forms preferably are bioequivalent, with respect to lopinavir, lamivudine and zidovudine, to a combination of two Kaletra tablet (200 mg lopinavir and 50 mg ritonavir per tablet) and one Combivir tablet (150 mg lamivudine and 300 mg zidovudine per tablet).

The first and second layers of a tablet dosage form of the invention can be prepared by a variety of techniques such as, without limitation, granulation (e.g., wet or dry granulation), blending, melt-extrusion, spray-drying, co-precipitation, freeze drying, or other solvent evaporation or solid dispersion techniques, with melt-extrusion and spray-drying being preferred for the preparation of the first layer.

Melt-extrusion and spray-drying are non-limiting examples that are suitable for making solid dispersions. Powdery solid dispersions can be, for example, directly compressed to form the first layer of a tablet dosage form of the invention. Non-powdery solid dispersions can be, for example, milled or ground to small particles before being compressed. Additional ingredients or excipients may be mixed with the solid dispersions before grinding and/or compression.

The melt-extrusion process typically comprises (1) preparing a melt which includes the active ingredient(s) (e.g., lopinavir and/or ritonavir), a pharmaceutically acceptable hydrophilic polymer as described above, and preferably a pharmaceutically acceptable surfactant as described above, and (2) cooling the melt until it solidifies. Melting often involves a transition into a liquid or rubbery state in which it is possible for one component to get dissolved or embedded, preferably homogeneously dissolved or embedded, in the other component or components such as the pharmaceutically acceptable hydrophilic polymer. In many cases, the polymer component(s) will melt and the other components including the active ingredient(s) and surfactant will dissolve in the melt thereby forming a solution. In such a case, the polymer functions as a solvent. Melting often involves heating above the softening point of the polymer. The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredient(s) efficiently. In addition, it may be convenient first to melt the polymer and then to mix in and homogenize the active ingredient(s). In one example, all materials except the surfactant are blended and fed into an extruder, while the surfactant is molten externally and pumped in during extrusion.

The melt temperature may range, for example, from 70 to 250° C., preferably from 80 to 180° C., most preferred from 100 to 140° C. Various additives may be included in the melt, for example, flow regulators such as colloidal silica; lubricants; fillers; disintegrants; plasticizers; or stabilizers such as antioxidants, light stabilizers, radical scavengers, or stabilizers against microbial attack.

In another example, the melt comprises lopinavir, ritonavir, or preferably both, as well as a pharmaceutically acceptable hydrophilic polymer as described above; and the melt temperature is in the range of from 100 to 170° C., preferably from 120 to 150° C., and highly preferably from 135 to 140° C. The melt can also include a pharmaceutically acceptable surfactant as described above.

In still another example, the melt comprises lopinavir, ritonavir, and a pharmaceutically acceptable polymer as described above. The melt can also include a pharmaceutically acceptable surfactant as described above. The melt temperature can be in the range of from 100 to 170° C., preferably from 120 to 150° C., and highly preferably from 135 to 140° C.

To start a melt-extrusion process, the active ingredient(s) (e.g., lopinavir and/or ritonavir) can be employed in their solid forms, such as their respective crystalline forms. The active ingredient(s) can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

The melting and/or mixing can take place in an apparatus customary for this purpose. Particularly suitable ones are extruders or kneaders. Suitable extruders include single screw extruders, intermeshing screw extruders or multiscrew extruders, preferably twin screw extruders, which can be corotating or counterrotating and, optionally, be equipped with kneading disks. It will be appreciated that the working temperatures will be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt can range from thin to pasty to viscous. Shaping of the extrudate can be conveniently carried out, for example, by a calender with two counter-rotating rollers with mutually matching depressions on their surface. The extrudate can be cooled and allow to solidify. The extrudate can also be cut into pieces, either before (hot-cut) or after solidification (cold-cut).

The solidified extrusion product can be further milled, ground or otherwise reduced to granules or particles with desired sizes. The solidified extrudate, as well as each granule or particle produced, comprises a solid dispersion, preferably a solid solution or glassy solution, of the active ingredient(s) in a matrix comprised of the hydrophilic polymer and optionally the surfactant. Where the granules/particles do not contain any surfactant, a pharmaceutically acceptable surfactant described above can be, for example, added to and blended with the granules/particles. The extrusion product can also be blended with other active ingredient(s) and/or additive(s) before being milled or ground to granules/particles. The granules/particles can be, for example, further processed and compressed to form the first layer of a tablet dosage form of the invention. The second layer material may be compressed simultaneously or sequentially with the first layer to form a bilayer tablet.

In some cases, direct-shaping techniques such as injection moulding can be used in combination with melt extrusion to prepare the first layer of a tablet dosage form of the invention.

In one example, copovidone and one or more surfactants are mixed and granulated, followed by the addition of aerosil and the active ingredient(s) (e.g., lopinavir, ritonavir, or preferably a combination of lopinavir and ritonavir). The mixture, which may contain for example at least 5% by weight of the active ingredient(s) is then milled. The mixture is then subject to extrusion, and the extrudate thus produced can be milled and sieved for further processing for the preparation of the first layer. Surfactant(s) employed in this example can also be added through liquid dosing during extrusion.

The approach of solvent evaporation, via spray-drying, may provide the advantage of allowing for processability at lower temperatures, if needed, and allow for other modifications to the process in order to further improve powder properties. In many cases, the spray-dried powder can be formulated further, if needed, and then compressed to form the first layer of a tablet dosage form of the invention.

Exemplary spray-drying processes and spray-drying equipment are described in K. Masters, Spray Drying Handbook (Halstead Press, New York, 4^{th} ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus. This help to ensure that the dried droplets are essentially solid and can form a fine powder and do not stick to the apparatus wall. The spray-dried product can be collected by removing the material manually, pneumatically, mechanically or by other suitable means. The actual length of time to achieve the preferred level of dryness can depend on the size of the droplets, the formulation, and spray dryer operation. Following the solidification, the solid powder may stay in the spray drying chamber for additional time (e.g., 5-60 seconds) to further evaporate solvent from the solid powder. The final solvent content in the solid dispersion as it exits the dryer is preferably at a sufficiently low level so as to improve the stability of the final product. For instance, the residual solvent content of the spray-dried powder can be less than 2% by weight. Highly preferably, the residual solvent content is within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other processes.

Like the solid extrudate described above, the spray dried product can contain a solid dispersion, preferably a solid solution and more preferably a glassy solution, of the active ingredient(s) in a matrix comprised of a pharmaceutically acceptable hydrophilic polymer as described above and optionally a pharmaceutically acceptable surfactant as described above. Where the spray dried product does not contain any surfactant, a pharmaceutically acceptable surfactant described above may be optionally added to and blended with the spray-dried product before further processing.

Before feeding into a spray dryer, the active ingredient(s) (e.g., lopinavir, ritonavir, or preferably a combination of ritonavir and lopinavir), a pharmaceutically acceptable hydrophilic polymer described above, and optionally a pharmaceutically acceptable surfactant described above can be dissolved in a solvent. Suitable solvents include, but are not limited to, water, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

A solid dispersion (preferably a solid solution or a glassy solution) prepared by melt-extrusion, spray-drying or other techniques may include both lopinavir and ritonavir. Ritonavir solid dispersion and lopinavir solid dispersion can also be separately prepared as described above, optionally milled, blended, and then compressed into the first layer. In one embodiment, ritonavir solid dispersion described above and lopinavir solid dispersion described above are separately prepared, and compressed into two different layers in the first layer of a tablet dosage form of the invention.

The first layer of a tablet dosage form of the invention can also comprise one or more additives, such as flow regulators, binders, lubricants, fillers, disintegrants, or plasticizers. These additives can be, for example, mixed with milled or powdery solid dispersions before compression. These additives can also be, for example, mixed with solid dispersions before milling. Disintegrants may promote a rapid disintegration of a tablet in the stomach and keep the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell® or Emcocel®), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil®). Non-limiting examples of suitable lubricants include those described above, such as magnesium and calcium stearates, sodium stearyl fumarate, and the like.

The second layer of a tablet dosage form of the invention can be prepared using techniques that are suitable for formulating the other therapeutic agent comprised in the second layer. Non-limiting examples of these techniques include blending, dry granulation, wet granulation, or solid dispersion. For instance, the other therapeutic agent (e.g., lamivudine, or a combination of lamivudine and zidovudine) can be blended with suitable excipients, milled, and then compressed with the first layer to form a bilayer tablet core. Other additives described above may also be included in the second layer.

Preferably, the weight ratio of the second layer to the first layer is no more than 1:3, such as no more than 1:4, 1:5, or 1:6. Techniques suitable for compressing two layers together in a tablet are known in the art. In some cases, the first layer can be pre-compressed before the compression of the second layer.

In order to facilitate the intake of a tablet dosage form, it is advantageous to give the tablet an appropriate shape. Large tablets that can be swallowed comfortably are therefore preferably elongated rather than round in shape.

A film coat on the tablet core further contributes to the ease with which it can be swallowed. A film coat also improves taste and provides an elegant appearance. The film-coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. polysorbates, and optionally a pigment, e.g. titanium dioxide or iron oxides. The film-coating may also comprise talc as anti-adhesive. Preferably, the film coat accounts for less than 5% by weight of a tablet dosage form of the present invention. In one embodiment, a tablet dosage form of the invention (e.g., with a bilayer tablet core described herein) is coated with Opadry® HPMC based coating solution.

Various other additives may also be used in preparing a tablet dosage form of the invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack.

In another aspect, the present invention features tablet dosage forms suitable for pediatric uses. Each tablet dosage form of the invention described hereinabove has a corresponding pediatric tablet dosage form, where the pediatric tablet has the same active ingredients and excipients as the regular tablet, as well as the same mean particle size, except that each component in the pediatric tablet is only one half of the amount of the same component in the regular tablet. For instance, a pediatric tablet dosage form of the invention can comprise a first layer and a second layer, wherein the first layer comprises (i) 100 mg lopinavir, (ii) 25 mg ritonavir, (iii) a pharmaceutically acceptable hydrophilic polymer as described above, and (iv) a pharmaceutically acceptable surfactant as described above; and the second layer comprises another therapeutic agent (e.g., 37.5 mg lamivudine, or a combination of 37.5 mg lamivudine and 75 mg zidovudine).

The weight ratio of the second layer to the first layer in a pediatric tablet dosage form of the invention can be, for example and without limitation, no more than 1:4. Preferably, the weight ratio of the second layer to the first layer is no more than 1:5; for example, the weight ratio of the second layer to the first layer can be 1:5, 1:6 or less. Also preferably, the first layer comprises at least 60% weight of the tablet dosage form. More preferably, the first layer comprises at least 70% weight of the tablet dosage form. Highly preferably, the first layer comprises at least 80% weight of the tablet dosage form.

A pediatric tablet dosage form of the invention can be, for example and without limitation, from 0.55 g to 0.85 g, and the first layer can be, for example and without limitation, from 0.45 g to 0.65 g. For instance, the tablet dosage form can be from 0.55 g to 0.75 g, and the first layer is from 0.45 g to 0.65 g. For instance, the tablet dosage form can be from 0.6 g to 0.7 g, and the first layer is from 0.5 g to 0.6 g. In one example, the tablet dosage form is no more than 0.85 g, and the first layer is at least 0.5 g. In another example, the tablet dosage form can be no more than 0.8 g, and the first layer is at least 0.5 g. In another yet example, the tablet dosage form can be no more than 0.75 g, and the first layer is at least 0.5 g. In yet another example, the tablet dosage form is no more than 0.7 g, and the first layer is at least 0.5 g. In yet another example, the tablet dosage form is no more than 0.85 g, and the first layer is at least 0.55 g. In yet another example, the tablet dosage form is no more than 0.8 g, and the first layer is at least 0.55 g. In yet another example, the tablet dosage form is no more than 0.75 g, and the first layer is at least 0.55 g. In yet another example, the tablet dosage form is no more than 0.7 g, and the first layer is at least 0.55 g. In yet another example, the tablet dosage form is no more than 0.85 g, and the first layer is at least 0.6 g. In yet another example, the tablet dosage form is no more than 0.8 g, and the first layer is at least 0.6 g. In yet another example, the tablet dosage form is no more than 0.75 g, and the first layer is at least 0.6 g. In yet another example, the tablet dosage form is no more than 0.85 g, and the first layer is from 0.5 g to 0.65 g. In another example, the tablet dosage form is no more than 0.85 g, and the first layer is at from 0.5 g to 0.6 g. In yet another example, the tablet dosage form is no more than 0.85 g, and the first layer is from 0.5 g to 0.55 g. In yet another example, the tablet dosage form is no more than 0.85 g, and the first layer is from 0.55 g to 0.65 g. In yet another example, the tablet dosage form is no more than 0.85 g, and the first layer is from 0.55 g to 0.6 g. In yet another example, the tablet dosage form is no more than 0.8 g, and the first layer is from 0.5 g to 0.65 g. In another example, the tablet dosage form is no more than 0.8 g, and the first layer is at from 0.5 g to 0.6 g. In yet another example, the tablet dosage form is no more than 0.8 g, and the first layer is from 0.5 g to 0.55 g. In yet another example, the tablet dosage form is no more than 0.8 g, and the first layer is from 0.55 g to 0.65 g. In yet another example, the tablet dosage form is no more than 0.8 g, and the first layer is from 0.55 g to 0.6 g. In yet another example, the tablet dosage form is no more than 0.75 g, and the first layer is from 0.5 g to 0.65 g. In another example, the tablet dosage form is no more than 0.75 g, and the first layer is at from 0.5 g to 0.6 g. In yet another example, the tablet dosage form is no more than 0.75 g, and the first layer is from 0.5 g to 0.55 g. In yet another example, the tablet dosage form is no more than 0.75 g, and the first layer is from 0.55 g to 0.65 g. In yet another example, the tablet dosage form is no more than 0.75 g, and the first layer is from 0.55 g to 0.6 g.

The first layer in a pediatric tablet dosage form of the invention preferably includes a solid dispersion comprising ritonavir, lopinavir, a pharmaceutically acceptable hydrophilic polymer described above, and a pharmaceutically acceptable surfactant described above. More preferably, the first layer comprises a solid solution comprising ritonavir, lopinavir, a pharmaceutically acceptable hydrophilic polymer described above, and a pharmaceutically acceptable surfactant described above. Highly preferably, the first layer comprises a glassy solution comprising ritonavir, lopinavir, a pharmaceutically acceptable hydrophilic polymer described above, and a pharmaceutically acceptable surfactant described above.

In a pediatric tablet dosage form of the invention, lopinavir and ritonavir can also be formulated, for example, in different solid dispersions and then mixed and included in the first layer. In one example, the first layer comprises a first and second solid dispersions, wherein the first solid dispersion comprises lopinavir and a first pharmaceutically acceptable hydrophilic polymer, and the second solid dispersion comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid dispersions, wherein the first solid dispersion comprises lopinavir, a first pharmaceutically acceptable hydrophilic polymer, and a first pharmaceutically acceptable surfactant, and the second solid dispersion comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In yet another example, the first layer comprises a first and second solid solutions, wherein the first solid solution comprises lopinavir and a first pharmaceutically acceptable hydrophilic polymer, and the second solid solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second solid solutions, wherein the first solid solution comprises lopinavir, a first pharmaceutically acceptable hydrophilic polymer, and a first pharmaceutically acceptable surfactant, and the second solid solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

In still another example, the first layer comprises a first and second glassy solutions, wherein the first glassy solution comprises lopinavir and a first pharmaceutically acceptable hydrophilic polymer, and the second glassy solution comprises ritonavir and a second pharmaceutically acceptable hydrophilic polymer, and the first layer also contains a pharmaceutically acceptable surfactant. The first and second hydrophilic polymers can be the same or different. In another example, the first layer comprises a first and second glassy solutions, wherein the first glassy solution comprises lopinavir, a first pharmaceutically acceptable hydrophilic polymer, and a first pharmaceutically acceptable surfactant, and the second glassy solution comprises ritonavir, a second pharmaceutically acceptable hydrophilic polymer, and a second pharmaceutically acceptable surfactant. The first and second hydrophilic polymers can be the same or different; and the first and second surfactants can also be the same or different.

Any other therapeutic agents that can be included in the second layer of a regular tablet dosage form of the invention can also be included in the second layer of a pediatric tablet dosage form of the invention. In one embodiment, the second layer of a pediatric tablet dosage form comprises 37.5 mg lamivudine. In another embodiment, the second layer of a pediatric tablet dosage form comprises 37.5 mg lamivudine and 75 mg zidovudine. In yet another embodiment, the second layer of a pediatric tablet dosage form comprises 100 mg raltegravir potassium (or the corresponding amount of raltegravir salt, e.g., 108.6 mg raltegravir potassium).

In yet another aspect, the present invention features methods of using a tablet dosage form of the invention to treat HIV infection. The methods comprise administering a tablet dosage form of the invention to a patient in need thereof. Any tablet dosage form described herein (including a pediatric tablet dosage) can be used in a method of the invention.

In any aspect, example, embodiment or preference described herein, lopinavir and ritonavir in the first layer of a tablet dosage form of the invention can be readily replaced with another therapeutic agent(s). Therefore, the present invention also features such tablet dosage forms as well as their corresponding pediatric tablet dosage forms.

It should be understood that the above-described embodiments and the following examples are given by way of illustration, not limitation. Various changes and modifications within the scope of the present invention will become apparent to those skilled in the art from the present description.

### Example 1

Lopinavir/ritonavir extrudate was prepared according to the process described in Example 2 of U.S. Pat. No. 8,025,899. The extrudate was milled to a mean particle size of 165 μm using standard milling equipment (Fitzmill or Alpine mill). A portion or all of the milled extrudate was combined with sodium stearyl fumarate and colloidal silicon dioxide and passed through a screen or comill and the mixture was blended. 3TC, sodium starch glycolate, sodium stearyl fumarate, colloidal silicon dioxide and part or all of the microcrystalline cellulose were passed through a screen or comill and then blended. The lopinavir/ritonavir blend (1220 mg; the first layer) and the 3TC blend (210 mg; the second layer) are compressed into a bilayer tablet using an automated bilayer tablet press. The composition of the bilayer tablet so prepared is provided in Table 1. The tablets thus prepared showed no or significantly less cracking. The tablets were then dedusted and coated using an HPMC-based aqueous coating suspension.

TABLE 1
Lopinavir/Ritonavir (LPV/RTV) Layer

| Milled LPV/RTV Extrudate (mg) | 1199.7 (200 mg lopinavir, |
|---|---|
| | 50 mg ritonavir)) |
| Sodium Stearyl Fumarate (mg) | 12.3 |
| Silicon Dioxide Colloidal (mg) | 8 |
| Total LPV/RTV Layer (mg) | 1220 |

Lamivudine (3TC) Layer

| | |
|---|---|
| 3TC (mg) | 75 |
| Macrocrystalline Cellulose (mg) | 126.6 |
| Sodium Starch Glycolate (mg) | 6.3 |
| Silicon Dioxide Colloidal (mg) | 0.4 |
| Sodium Stearyl Fumarate (mg) | 1.7 |
| Total 3TC Layer (mg) | 210 |

### Example 2

Lopinavir/ritonavir extrudate was prepared according to the process described in Example 2 of U.S. Pat. No. 8,025,899. The extrudate was milled to a mean particle size of 140 μm using standard milling equipment (Fitzmill or Alpine mill). A portion or all of the milled extrudate was combined with sodium stearyl fumarate and colloidal silicon dioxide and passed through a screen or comill and the mixture was blended. AZT, 3TC, sodium starch glycolate, sodium stearyl fumarate, colloidal silicon dioxide and the microcrystalline cellulose were screened and blended. The lopinavir/ritonavir blend (1220 mg; the first layer) and the AZT/3TC blend (300 mg; the second layer) are compressed into a bilayer tablet using an automated bilayer tablet press. The composition of the bilayer tablet so prepared is provided in Table 2. The tablets thus prepared showed no or significantly less cracking. The tablets were then dedusted and coated using an HPMC-based aqueous coating suspension.

TABLE 2
Lopinavir/Ritonavir (LPV/RTV) Layer

| Milled LPV/RTV Extrudate (mg) | 1199.7 (200 mg lopinavir, |
|---|---|
| | 50 mg ritonavir)) |
| Sodium Stearyl Fumarate (mg) | 12.3 |
| Silicon Dioxide Colloidal (mg) | 8 |
| Total LPV/RTV Layer (mg) | 1220 |

Zidovudine/Lamivudine (AZT/3TC) Layer

| | |
|---|---|
| AZT (mg) | 150 |
| 3TC (mg) | 75 |
| Macrocrystalline Cellulose (mg) | 69.1 |
| Sodium Starch Glycolate (mg) | 2.3 |
| Silicon Dioxide Colloidal (mg) | 0.9 |
| Sodium Stearyl Fumarate (mg) | 2.7 |
| Total AZT/3TC Layer (mg) | 300 |

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

1. A tablet dosage form comprising a first layer and a second layer, wherein the first layer comprises compressed solid dispersion particles each of which comprises ritonavir and lopinavir in a solid dispersion, said solid dispersion comprising a pharmaceutically acceptable hydrophilic polymer and a pharmaceutically acceptable surfactant, and said solid dispersion particles having a mean particle size of no more than 200 μm, and wherein said second layer comprises another therapeutic agent.

2. The tablet dosage form of claim 1, wherein the weight ratio of the second layer to the first layer is no more than 1:2.

3. The tablet dosage form of claim 1, wherein the weight ratio of the second layer to the first layer is no more than 1:3.

4. The tablet dosage form of claim 1, wherein the weight ratio of the second layer to the first layer is no more than 1:4.

5. The tablet dosage form of claim 1, wherein the dosage form is no more than 1.6 g, and the first layer is at least 1 g.

6. The tablet dosage form of claim 1, wherein the dosage form is no more than 1.6 g, and the first layer is at least 1.1 g.

7. The tablet dosage form of claim 1, wherein the dosage form is no more than 1.6 g, and the first layer is at least 1.2 g.

8. The tablet dosage form of claim 1, wherein the dosage form is from 1.4 to 1.6 g, and the first layer is from 1.1 to 1.3 g.

9. The tablet dosage form of claim 8, wherein said first layer comprises 200 mg lopinavir and 50 mg ritonavir, and said second layer comprises 75 mg lamivudine.

10. The tablet dosage form of claim 9, wherein said second layer further comprises 150 mg zidovudine.

11. The tablet dosage form of claim 9, wherein said solid dispersion particles have a mean particle size of from 120 to 190 μm.

12. The tablet dosage form of claim 1, wherein said first layer comprises 100 mg lopinavir and 25 mg ritonavir, and said second layer comprises 37.5 mg lamivudine.

13. The tablet dosage form of claim 11, wherein said second layer further comprises 75 mg zidovudine.

14. A tablet dosage form according to claim 1, wherein said pharmaceutically acceptable hydrophilic polymer has a Tg of at least 50° C.

15. A tablet dosage form according to claim 1, wherein said solid dispersion is a solid solution.

16. A tablet dosage form according to claim 1, wherein said solid dispersion is a glassy solution.

17. A tablet dosage form according to claim 1, wherein said pharmaceutically acceptable hydrophilic polymer has a Tg of at least 50° C., and said pharmaceutically acceptable surfactant has an HLB value of from 4 to 10.

18. A tablet dosage form according to claim 1, wherein said pharmaceutically acceptable hydrophilic polymer is copovidone, and said pharmaceutically acceptable surfactant is sorbitan monolaurate.

19. A process of making a tablet dosage form according to claim 1, comprising compressing said first layer and said second layer.

20. A bottle of tablets according to claim 1, wherein no more than 10% of said tablets show cracking.

21. The bottle of claim 20, wherein no more than 5% of said tablets show cracking.
